(19) United States
(12) Patent Application Publication
(10) Pub. No.: US 2008/0262404 A1
(43) Pub. Date: Oct. 23, 2008

(54) Gantry jockstrap

(76) Inventor: Ann McKenzie, Santa Cruz, CA (US)

Correspondence Address: DERGOSITS & NOAH LLP, FOUR EMBARCADERO CENTER, SUITE 1450, SAN FRANCISCO, CA, 94111, US

(21) Appl. No.: 11/789,213

(22) Filed: Apr. 23, 2007

(51) Int. Cl. A63B 71/08; A61F 5/40

(52) U.S. Cl. 602/70

(57) ABSTRACT

An undergarment for men providing an improved method of support for male genitalia during physical activity. The design comprises a wider pouch, a lowered V-waistband and a unique cross over sewing pattern for the leg straps which provide increased support and comfort for the wearer especially during activity. All parts of the design are made using a single type of cotton-lycra or microfiber fabric.

### TECHNICAL FIELD

This invention relates generally to an undergarment for men, and particularly to a new and innovative method of support for male genitalia during periods of physical activity in the form of improved athletic supporter or jockstrap. The individual and combined design elements can be applied to a variety of undergarment needs and applications, providing a high level of support, comfort and protection.

### BACKGROUND OF THE INVENTION

Athletic supporters are widely used by males during athletic activities and after certain medical procedures, such as hernias and vasectomies to provide support and protection to the genitalia. This type of supporter generally consists of a narrow, elongated knitted pouch, attached to a wide waistband, and leg straps which extend from the bottom of the pouch at the crotch around the legs attaching at the waistband. With the exception of improvements in materials, the basic design has changed minimally, although the need for support has increased due to changes in sports activities and additional medical uses.

In the prior art, the most supportive elements of the design are the waistband and leg straps which are made of a material holding a knit pouch of minimally supportive material. One of the consistent problems is the use of at least two materials differing in composition, weight and stretch characteristics. The difference in deterioration rates of the materials used combined with stress at the joining points result in a shortened life span and use of the product.

The most typical athletic supporter consists of a wide band that sits high on the waist and is the attachment point for the pouch and leg straps. Although jockstraps are typically made in this manner, the wide band at the waist is subject to rolling over due to activity and/or body type. For certain medical situations requiring genital support, such as a hernia, the high waistband would cause discomfort by being situated on or near the affected area. Additionally, the leg strap attachments to the waistband add seams and additional fabric layers which can cause pressure at the pelvic bones.

Compression shorts and other close fitting briefs have taken the place of athletic supporters in many cases, but do not provide an equivalent level of support in that the genitals are held by compression to the body. An athletic supporter is able to provide specific genital support by holding the genitals securely in a pouch that is independent from the body.

The two most commonly worn jockstraps currently are made by Bike and Champion.

Bike's jockstrap has a wide waistband that sits high on the waist. The design of the waistband is a straight cut versus being ergonomically cut and sewn to follow a man's body shape. The straight cut results in roll-over during activity or sitting. The pouch is made with a single vertical stretch which provides no lateral support for the genitals during movement. The pouch is sewn at a right angle and the seaming and shaping does not follow the contours of the male anatomy or provides sufficient room or support for the genitals.

The attachment point of the leg straps to the pouch at the bottom portion of the pouch is a problem with all prior jockstrap designs. The dart where the leg straps join at the center bottom of the pouch is a point of stress given the different fabrics used and the layers sewn on top of each other. This creates a hard knot of fabric at a particularly sensitive area of the body. This part of the jockstrap is the most highly stressed point structurally and in the prior art subject to additional stress caused by different rates of material deterioration, stitching and as a single attachment point, frays easily and pulls apart.

The straps are made from a separate material, usually, which is subject to earlier deterioration of the rubber being used. Where the leg straps attach to the waistband, the resulting bulk forms knots that push into the hip joint. Further, the placement of the attachment points of the leg straps on the sides of the waistband provides no lateral support for the pouch.

The Champion jockstrap has some minor changes to the basic design but still does not address the main problems with currently used jockstraps. The waistband, though narrower, is cut in a straight line and not angled to conform to the body.

The waistband is fabric covered, but still creates bulk of material along the hip joint. The Champion jockstrap has an added dart at the bottom of the pouch but is still cut at a right angle and does not conform to genitalia. While the sides of the pouch are bound, the binding does not provide stability due to the non-supportive nature of the material being used. The attachment points at the waistband and crotch have multiple fabric layers and offer no structural advantage with the addition of bar tacking. The location of the leg attachment points at the hip joints is too far out on the waistband to provide any support to the wearer.

Other jockstrap designs have implemented various design elements which attempt to improve the support and comfort of the athletic supporter for the wearer.

One such prior art garment is shown in U.S. Pat. No. 4,453,541 to Castelli which discloses an athletic supporter which has a high, wide waistband and leg straps which terminate at a single point at the bight. The high waisted band is subject to roll over and the termination at a single point provides only lateral support.

U.S. Pat. No. 5,003,972 to Kestler is an adjustable athletic supporter with velcro or a series of snaps at the waistband to allow for adjustable fitting of the pouch for sizing and support due to wear and material deterioration. The waistband is wide and high to accommodate the adjustability of the pouch to improve fit and longevity of the product but does not answer the necessity to provide a real improvement in support for genitalia. The leg straps are still connected at a single point at the bight which results only a lateral support during activity.

U.S. Pat. No. 4,679,554 to Markham uses tubular construction on the waistband and leg straps as well as a single material for the construction of the supporter. However, the pouch shape is still vertical and the leg straps are sewn on top of the high waistband, adding additional layers of material at the pelvic bone point of movement at the hips. The modification to the waistband using a tubular fabric design instead of the wide is still subject to the same rolling and twisting due to the same high waist design. The same single attachment point at the crotch is subject to additional stress of physical activity due to a single central point of attachment to the pouch.

All of the above mentioned patents are directed to the comfort and support of the wearer and are attempts to solve the problems associated with wearing athletic supporters. However, each of the prior art improvements are restricted by their use of conventional shapes, structure and materials. Merely changing the materials used or changing a single portion of the design is insufficient to correct the problems with the prior art athletic supporters.

### SUMMARY OF THE INVENTION

The present invention involves an athletic support garment comprising a V-shaped waistband sized to reside below the waist of a user when the athletic support garment is so worn. The athletic support garment includes an pouch having a substantially linear top edge affixed to said V-shaped waistband and diagonally extending edges emanating from the substantially linear top edge and meeting at a point distal from it. A pair of straps are affixed to the V-shaped waistband at their first ends at the extreme ends of the substantially linear top edge. Each strap is joined to the pouch along the diagonally extending edges. Support is enhanced whereby the straps cross over one another at the distal point. The straps then join the waistband at their second ends substantially at points at which the first ends join the waistband.

### BRIEF DESCRIPTION OF THE FIGURES

FIG. 1 is a perspective front view of the athletic support garment of the presentation invention.

FIG. 2 is a side plan view of the athletic support garment of FIG. 1.

FIG. 3 is a bottom plan view of the athletic support garment of FIG. 1.

### DETAILED DESCRIPTION OF THE INVENTION

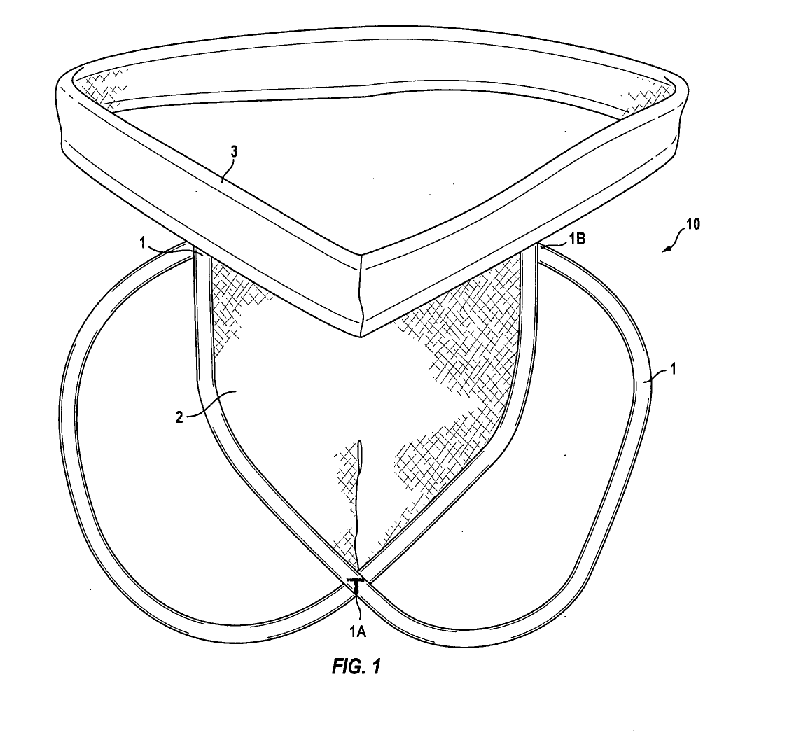

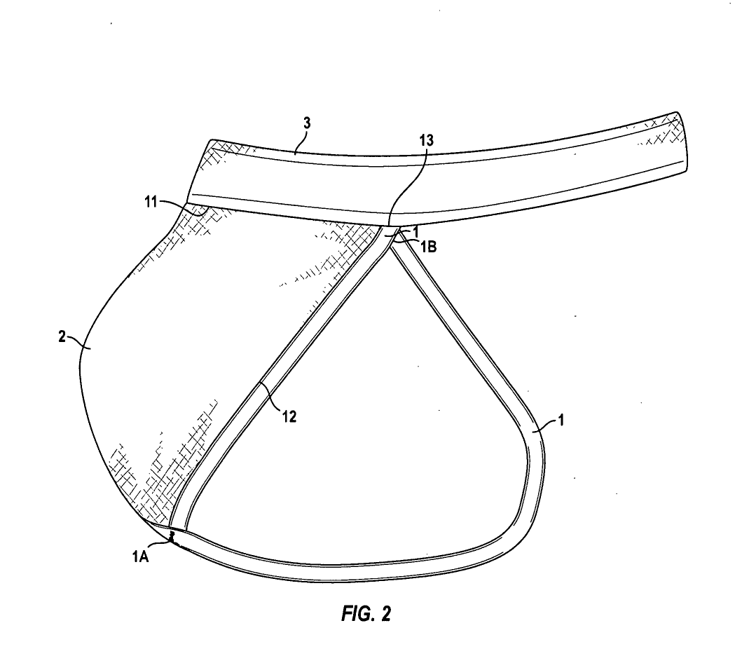

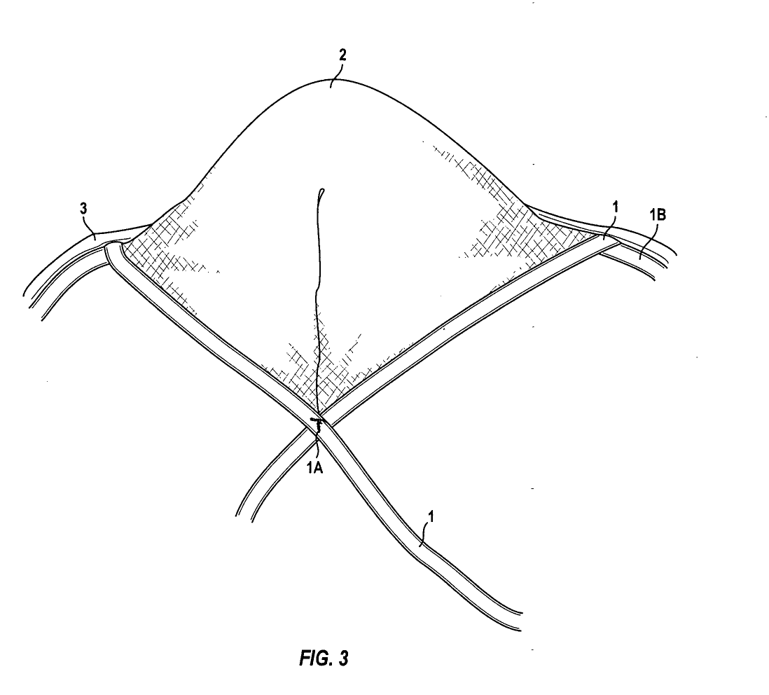

The present invention involves athletic support garment 10 depicted in FIGS. 1-3. Specifically, athletic support garment 10 comprises V-shaped waistband 3 sized to reside below the waist of a user when said athletic support garment is so worn. A pouch 2 is characterized as having linear top edge 11 affixed to V-shaped waistband 3 and diagonally extending edges 12 emanating from substantially linear top edge 11 and meeting at a point 1A distal from substantially linear top edge 11. A pair of straps 1 are affixed to V-shaped waistband 3 at first ends 13 which is also at the extreme ends of substantially linear top edge 11. Each strap 1 is joined to pouch 2 along said diagonally extending edges 12 joining waistband 3 at their second ends 1B shown as being substantially at points which said first ends 13 join said waistband. (As noted, athletic support garment 10 is characterized whereby straps 1 cross over one another at the point where diagonally extending edges 12 join one another at 1A.)

Ideally, athletic support garment 10 and all of its component parts, namely, V-shaped waistband 3, pouch 2 and straps 1 are all composed of the same material. This material is ideally a member selected from the group consisting of cotton/lycra and microfibers.

The present athletic support garment 10 is ergonomically designed to provide an improved level of support and comfort to a wearer by the combined use of an innovative design wherein component parts in the form of pouch 2, waistband 3 and straps 1 co-act with one another synergistically. As noted above, all of these parts are preferably composed of a single material establishing an improved level of comfort and support while resisting premature wear. Ideally, all components of the garment are made of stretch fabric such as 55% cotton, 35% polyester and 10% Lycra Spandex composite.

It is noted, particularly in reference to FIG. 1, that by extending pouch 2 to a width whereby diagonal edges 12 are sewn to straps 1 increases comfort and support. This is further enhanced by the cross over design of straps 1 as shown. A protective cup can also be used in conjunction with the present invention while remaining within the scope thereof.

It should also be noted that V-shaped waistband 3 is improved over prior art designs for both sports and medical considerations. The waistband is designed to sit below the waist of a user minimizing clothing binding. In the instance of hernia surgery recovery, V-shaped waistband 3 would be positioned not to interfere with the point in incision. In the case of a vasectomy, the widened pouch design provides more comfort and support during recovery. The present invention is also useful in providing stable support for cooling packs now used in male fertility treatments.

In providing a garment of a single cotton/lycra or microfiber stretch material, the problems of differing rates of deterioration between rubber contained in s and the materials themselves used in the pouch is lessened. Single suppliers can now also be employed to make the complete athletic support garment not requiring components from one-supplier and non-components from a second.

In summary, the present invention involves an athletic support garment which provides a level of comfort, support and wearability unmatched by prior designs. Although a preferred embodiment of the present invention has been described above, it is recognized that the present invention is to be limited only by the claims which follow.

What is claimed is:

1. An athletic support garment comprising a V-shaped waistband sized to reside below the waist of a user when said athletic support garment is so worn, an pouch having a top edge affixed to said V-shaped waistband and diagonally extending edges emanating from said top edge and meeting at a point distal from said top edge, a pair of straps affixed to said V-shaped waistband at their first ends at the extreme ends of said top edge, each strap being joined to said pouch along said diagonally extending edges crossing over at the distal point and joining said waistband at second ends thereof substantially at points at which said first ends join said waistband.

2. The athletic support garment of claim 1 wherein said straps cross over one another substantially at the joining of the diagonally extending edges of said pouch.

3. The athletic support garment of claim 1 wherein said V-shaped waistband, pouch and pair of straps are all composed the same material.

4. The athletic support garment of claim 2 wherein said material comprises a member selected from the group consisting of cotton/lycra and microfibers.
